(19) United States
(12) Patent Application Publication
(10) Pub. No.: US 2012/0123356 A1
(43) Pub. Date: May 17, 2012

(54) FOAM MATERIAL FOR MEDICAL USE AND METHOD FOR PRODUCING SAME

(75) Inventor: Bryan GREENER, York (GB)

(73) Assignee: Smith & Nephew Plc, London (GB)

(21) Appl. No.: 13/332,814

(22) Filed: Dec. 21, 2011

Related U.S. Application Data
(62) Division of application No. 12/596,831, filed on Oct. 20, 2009.

(30) Foreign Application Priority Data
Apr. 21, 2007 (GB) 0707758.9

(51) Int. Cl. A61M 37/00; A61M 1/00

(52) U.S. Cl. 604/290; 604/313

(57) ABSTRACT

An in situ forming foam for medical applications and a method for making same is described, the method comprising the steps of: preparing a first component, Composition A, comprising an acidic solution of a polycationic polymer selected from the group comprising polymeric amines and polysaccharides; preparing a second component, Composition B, selected from the group comprising a metal carbonate, a metal bicarbonate or a mixture of a metal carbonate and a metal bicarbonate; maintaining said first and second components separately prior to mixing; and mixing said first and second components at an intended site of application. The foam is a mechanically robust but flexible and resilient material wherein the degree and nature of the porosity may be controlled.

The present invention relates to a foamed material suitable for use in medical applications such as the treatment of wounds, for example, and which foam may be generated in situ.

Up to the present time the commercial availability and success of foam materials formed in situ in a wound, for example, has been seriously limited by toxicity concerns and the poor physical performance of the resulting materials.

The possibility of producing a foamed material in situ relies on two key transformations: (1) the delivery and formation of a physically coherent polymeric structure; and (2) the foaming of this structure by gaseous blowing. These are also the constraints when producing a foam of any kind, but in addition, for medical applications, these steps must be achieved in the absence of a toxic species that could damage the biological environment, including proteinaceous tissues. This is difficult to achieve with currently available systems because the majority rely upon in situ polymerisation at delivery (eg, DIY polyurethane foam fillers). in situ polymerisation occurs when one or more monomers or prepolymers are combined at application, commonly in the presence of a catalytic initiator; these reactive species can also react indiscriminately with materials in contact with them, causing collateral damage. Foaming systems that do not reply upon the in situ preparation of a polymer can deliver a polymer in a propellant from a pressurised canister (eg, shaving foam), however the mechanical properties of these foams are not appropriate for load-bearing medical applications.

In situ foams have been the subject of limited inventive prior art. Notably, none of the prior art proposals have emerged commercially. The majority of inventions concern the in situ formations of alginate-based objects. Alginates are commonly applied medical materials and form self-supporting objects when formed. Alginates do not require polymerisation from monomeric species at application: sodium alginate is water-soluble and is semi-solidified by complexation with calcium (or other divalent metal) ions. At this stage, an additional reagent is required to achieve the foaming of this gel. Foams produced in this manner have limited mechanical properties and can easily be disrupted under light pressure, for example, surface mechanical loads of less than 40 g/cm or light mechanical distress are able to permanently deform and break up prior art foams.

The prior art discloses the preparation of polysaccharide-based foams (U.S. Pat. No. 5,840,777, U.S. Pat. No. 5,089,606) but these foams rely on ionic cross-linking (eg, alginate salts) and the introduction of a gas (eg, by beating) into an aqueous solution of polysaccharide for initial foaming. To form stable articles, these materials require drying.

The prior art discloses other non-foam ionic cross-linking polymerisations (U.S. Pat. No. 6,391,294) for in situ solidification involving the complexation of cations and anions at the site of application.

The prior art also discloses in situ forming polyurethane-based foams that are produced from isocyanate prepolymers (U.S. Pat. No. 5,064,653). These materials are suitable for in situ medical applications because of the hazardous nature of isocyanates.

In one instance, there is a need for an in situ forming foam for use in the management and treatment of battlefield injuries where wounds may be extensive and of particular types such as entry and exit wounds, where conventional flat sheet dressing materials are not suitable, and minimum handling or additional distress to the patient is desirable.

From the above disadvantages described with reference to the prior art it will be apparent that there is a need for an in situ forming foam which does not have toxic effects with respect to human tissue, is robust and which may be applied with a minimum of difficulty and process steps so as to minimise any additional trauma or distress to a patient.

According to a first aspect of the present invention there is provided a method of making a foam material, the method comprising the steps of preparing two separate constituents designated as Composition A and Composition B, wherein Composition A comprises an acidic solution of a polycationic polymer selected from the group comprising polymeric amines and polysaccharides and Composition B comprises a component selected from the group comprising metal carbonates, metal bicarbonates, and mixtures of metal carbonates and bicarbonates, said Compositions A and B being mixed together and upon reaction therebetween forms said foam material.

Preferably, the polysaccharide is chosen from a chitin derivative such as chitosan, for example, or from a chitosan derivative.

It should be understood that whilst the present invention comprises two reactive constituents, Compositions A and B, the further inclusion of additional ingredients to assist in formulation, mixing rather than as active pharmaceuticals (other than as further discussed hereinbelow) is not precluded.

The first aspect of this invention provides a method of making a homogeneous, substantially water insoluble but water absorbent polysaccharide foam at a site of application for medical use, for example.

According to a second aspect of the present invention there is provided a foam when made by the method of the first aspect of the present invention.

The aforementioned foam is produced by the combination of: the first compound, Composition A, which is an acidic solution of a neutral pH-insoluble polycationic polymer, formed from the polycationic polymer and at least one water-soluble acid, where the acid may or may not be covalently attached to the polymer backbone; Composition A being mixed with Composition B, which is a metal carbonate or bicarbonate or a composition including a metal carbonate or bicarbonate.

In the present invention it should be noted that pH 7.4 in relation to the human body is considered to be neutral whereas in strict chemical terms pH 7 is regarded as neutral with lower numbers being acidic and higher numbers being alkaline. In the context of the present invention, neutral pH means pH 7, not pH 7.4, since the following discussions are in the context of Composition A or the result of mixing Compositions A and B. Synthetic simulants of tissue fluid and blood are buffered to pH 7.4. However, in wounds, the actual pH observed can vary quite broadly depending upon aetiology.

The invention requires Composition A to be of sufficient acidity to protonate sufficient amine groups on the polycation to enable solubilisation. For the range of formulation concentrations given herein and the amine exemplified here (chitosan), acetic acid (pKa 4.76) provides a sufficiently acidic environment to enable efficient protonation and therefore solubilisation. To ensure that the viscosity of Composition A is sufficiently low to allow efficient mixing in the mixing head, a pH lower than the absolute solubility threshold of the polycation is desirable. This also affords a wide pH range separating the pH of Composition A from the neutral (pH 7) pH at which the polycation becomes de-solubilised. This is advantageous because it affords a conveniently broad formulation operating window during manufacture.

Composition A may have a pH below pH 7 and preferably has a pH below pH 6 and more preferably a pH below pH 5. For this purpose, the acid is preferably carboxylic in nature. For safety purposes the acid is also preferably an organic (eg, carboxylic) rather than an inorganic acid (eg HCl).

The acid may or may not be covalently bonded to the polymer backbone in Composition A.

The neutral pH-insoluble polycationic polymer is that which is insoluble at pH 7, preferably insoluble at any pH above pH 7, more preferably insoluble at any pH above pH 6. Examples of such polymers include polymeric amines, both synthetic and naturally derived. Preferably the polymer is a polysaccharide and is more preferably a chitin derivative, for example chitosan or a chitosan derivative that is insoluble at pH 7, preferably insoluble at any pH above pH 7, more preferably insoluble at any pH above pH 6.

In the embodiment of the invention where the acid is not covalently attached to the polymer backbone in Composition A, the water-soluble acid is preferably an organic carboxylic acid of the type R—COOH, where R can be any carbon-based organic moiety known to one skilled in the art. For safety purposes the acid is preferably chosen from the group of biologically acceptable organic acids that includes: acetic acid, lactic acid and glycolic acid, for example.

In an alternative embodiment of the invention where the acid is covalently attached to the polymer backbone in Composition A, the acidic functionalisation is preferably of the type-R—COOH, where R can be any carbon-based organic moiety known to one skilled in the art. A common methodology for such an acid functionalisation is the treatment of polysaccharides with a solution of chloroacetic acid, for example, or its salt sodium chloroacetate, so forming an ether link at polymer hydroxyl groups, resulting in carboxymethylation. Carboxymethylchitosan is an example of a polycation carrying covalent acidic functionalisation that renders the polymer water-soluble at neutral pH.

Composition B may consist entirely of solid metal carbonate or bicarbonate or may be a formulation of metal carbonate or bicarbonate. For ease of mixing at the site of application, Composition B is preferably a formulation of a metal carbonate or bicarbonate. More preferably, Composition B is a formulation of metal carbonate or bicarbonate in a water-miscible but substantially water-free liquid carrier. More preferably still, the metal carbonate or bicarbonate is insoluble in the water-miscible but substantially water-free liquid carrier (to avoid significant decomposition on storage). Even more preferably still, the water-miscible but substantially water-free carrier is of similar viscosity to Composition A when finally formulated, to enable effective mixing at the site of application. Examples of water-miscible but substantially water-free carriers of similar viscosity to Composition A when finally formulated include glycerol and poly(ethylene glycol).

Composition A may be formulated in any manner known in the art, for example by combining water with an acid and dissolving the polycation with stirring. In the embodiment where the acid is not covalently attached to the polymer, it is preferable to make up a stirred mixture of the polycation in water prior to the addition of the acid. In the alternative embodiment where the acid is covalently attached to the polymer, this material can simply be dissolved in water.

The composition of Composition A is not restricted by the invention, but preferably comprises polymer concentrations above 0.1% w/w, more preferably above 1% w/w of the formulation. An upper limit of polymer concentration may be about 20% w/w (threshold of solubility) as the viscosity becomes too high around this value.

Composition B may be formulated in any manner known to one skilled in the art, for example by combining metal carbonate or bicarbonate with the carrier with stirring.

The composition of Composition B is not restricted by the invention, but preferably comprises concentrations of metal carbonate or bicarbonate or mixtures thereof above 20% by mass, more preferably above 50% by mass of the formulation. Preferably, the upper limit of carbonate or bicarbonate concentration is below 90% by mass.

Compositions A and B can be stored in any acceptable manner prior to use. For convenient usage, Compositions A and B are preferably store loaded in a dual-barrelled syringe. The relative proportions by volume of Compositions A and B combined at the site of application are not restricted by the invention but are preferably in the volumetric ratio exceeding 1:1, more preferably exceeding 2:1, more preferably exceeding 4:1 and even more preferably exceeding 8:1 in favour of Composition A in each case.

Dual barrelled syringes with differential volume chambers offer a preferred method of dosing the relative proportions of Compositions A and B.

According to a third aspect of the present invention there is provided a method of making an in situ forming foam for use in medical applications, the method comprising the steps of:
preparing a first component, Composition A, comprising an acidic solution of a polycationic polymer selected from the group comprising polymeric amines and polysaccharides;
preparing a second component, Composition B, selected from the group comprising a metal carbonate, a metal bicarbonate or a mixture of a metal carbonate and a metal bicarbonate;
maintaining said first and second components separately prior to mixing; and
mixing said first and second components at an intended site of application.

In this specification, the term “in situ forming foam” means a foam which is formed in situ in a wound, or bodily cavity, for example, from the constituent components of the foam which are brought together and mixed at the intended site.

In a preferred embodiment of the method of the second aspect of the present invention, Composition A and Composition B are effectively both simultaneously mixed and applied to the intended site such as a wound, for example.

All of the discussion set out above relating to Composition A and Composition B in relation to the first and second aspects of the present invention are equally valid and applicable to this third aspect of the present invention.

In general terms the present invention concerns the in situ production of a mechanically robust foam for medical applications, for example in cavity filling and the replacement or augmentation of soft tissues including cartilage, ligaments and tendons. Wound repair, cartilage repair and bone repair are examples of some medical applications of this technology. The invention is of particular utility in the management of battlefield wounds, traumatic wounds and cavity wounds.

The in situ forming foam according to the present invention may be produced with either a closed cell structure or an open cell structure, the latter, rendering the foam both absorbent and able to transmit fluids, both gaseous and liquid, therethrough. Thus, the foam according to the present invention may advantageously be used as a porous cavity filler in combination or as an integral element with topical negative pressure (TNP) therapy, for example. As a very general statement, the foams produced according to the present invention are mechanically robust being flexible and resilient, i.e. able to be deformed and subsequently recover and having a nature much akin to a bath sponge. However, due to the ability to control the degree and nature of the porosity contained in the foam the range of mechanical properties is large.

The aim of this invention is the production of an in situ forming foam for medical applications. The objects are the absence of biologically incompatible species in the foam, in the pre-foam or in its intermediates and the economical use of pre-foam components.

It is known to those skilled in the art that chitosan is soluble in acidic media, including aqueous solutions. As discussed hereinabove, solubilisation can be achieved by providing an acid in solution or by covalently binding an acidic moiety to the polymer backbone (eg, by forming carboxymethylchitosan). In the present invention, either method of solubilisation is suitable.

The reaction of an acid with a metal carbonate, including higher carbonates such as bicarbonates, can result in neutralisation of the acid with concomitant liberation of carbon dioxide gas. A molar equivalent or excess of metal carbonate in Composition B to acid in Composition A ensures full neutralisation.

The two components may be stored separately prior to mixing at the site of application. Storage and mixing can be achieved by any means, but a dual barrelled syringe with static mixing head, as is known in the art, is preferred.

This system is economical and effective, comprising of a minimum of two ingredients other than water in the case where the acid is covalently linked to the polymer backbone.

When both components are mixed, the reaction of the metal carbonate with the acidic chitosan solution generates carbon dioxide gas in the process of neutralising the acid and solidifying the solubilised polymer, so achieving an objective of the invention, which is the neutralisation of the acid so as not to aggravate the wound site or cause further distress to the patient. The degree of foaming or blowing can be controlled independently of polymer solidification by utilisation of an appropriate quantity of metal carbonate and/or metal bicarbonate. Thus, the nature and extent of the pores in the foam material may be controlled.

At the site of application, Compositions A and B can be mixed by any method known to one skilled in the art, preferably by passage through a static mixing element. The static mixer is preferably attached to a double-barrelled syringe delivering both Compositions. The Compositions are delivered and mixed at a rate that allows the mixture to reach the site of application before significant foaming occurs. The applicator used to finally deliver the mixture to the intended site of application may be of any geometry, preferably a circular or near-circular orifice for the filling of cavities, preferably a “fish-tail” for the provision of a largely two-dimensional foamed slab. The applicator may have one or more outlets, depending upon application.

The second aspect of this invention is the use of the in situ formed foam (as described above) in medical applications. These applications include the management of traumatic wound cavities, including battlefield injuries, the filling of body cavities including any naturally occurring orifices or any sites of injury where there is a tissue void. These applications also include the in situ formation of topical wound dressings.

According to a fourth aspect of the present invention there is provided the use of an in situ forming foam according to the second aspect of the present invention for the treatment of wounds.

With these medical applications in mind, it should be clear that this invention also includes the use of the so-described foam materials for the inclusion and/or delivery of other therapeutic species such as antimicrobial species including antibiotics and antibacterials, pain-killers, growth factors, protease inhibitors, biological products and cells, for example. This includes the site of application co-mixing of these materials with Composition A or Composition B separately or when combined or at combination (for example using a triple barrelled syringe).

A particular embodiment of this invention is the use of chitosan-based Composition A formulated foams for the haemostatic management of battlefield injuries, particularly those caused by rapid tissue penetration and exit wounds. These wounds, particularly at exit, are not suited to management by a flat sheet intervention. Chitosan is a known haemostat and is currently being applied in this indication in flat sheet format.

Another particular embodiment of this invention is the use of the so-formed foam for the filling or part-filling of wound cavities prior to the application of negative pressure therapy. The foams are mechanically robust enough not to collapse under negative pressure in the region of −125 mmHg below atmospheric pressure, and at this pressure, for example, allows the transmission of liquid from wound bed to exit port. However, the in situ forming foams according to the present invention allows the transmission of fluids over a large range of negative pressures since the nature and size of the internal porosity may be controlled in the foaming process by selection of appropriate formulations and ratios of Compositions A and B.

A yet further particular embodiment of the present invention is the management of cavity wounds and the filling of traumatic wounds at the venue of injury where the in situ forming foam can be applied quickly and easily. On hospital admission, this foam can be removed from the trauma site before surgery, removing a substantial quantity of unwanted wound debris.

Another particular embodiment of this invention is the provision of the so-formed foam for internal void-filling applications, for example bone filling applications. The foam can be generated via an internally positioned mixing head (for example at the distal end of an endoscope or minimally invasive surgical tool).

Another particular embodiment this invention is the generation of minimally blown foams for the filling and/or repair of soft tissue surfaces, particularly the articulating surfaces associated with load-bearing joints including the hip, knee, ankle and shoulder.

Another particular embodiment of this invention is for the visualisation, by imprint casting, of tissue geometry abnormalities within a bodily orifice, particularly the colon.

Another particular embodiment of this invention is for the spatial filling of tissue voids or the expansion of tissue, for example in the remediation of spatial defects created during excision surgeries (eg, tumour removal) or traumatic injuries. This embodiment is intended to include plastic surgical procedures and cosmetic enhancements, for example to the soft tissues of the face including nose, cheeks, chin and lips.

According to a fifth aspect of the present invention there is provided a pharmaceutical composition comprising Composition A and Composition B as defined hereinabove.

According to a sixth aspect of the present invention there is provided a pharmaceutical composition comprising Composition A and Composition B, as defined hereinabove, for use in therapy.

The therapy of the sixth aspect includes but is not limited to the treatment of wounds and haemorrhage.

According to a seventh aspect of the present invention there is provided the use of Composition A and Composition B sequentially or in combination for the manufacture of a medicament for therapy.

The therapy of the seventh aspect includes but is not limited to the treatment of wounds and haemorrhage.

According to an eighth aspect of the present invention there is provided a chitosan- based in situ forming foam for therapy.

The therapy of the eighth aspect includes but is not limited to the treatment of wounds and haemorrhage.

According to an ninth aspect of the present invention there is provided the use of a chitosan-based in situ forming foam for the manufacture of a medicament for therapy.

The therapy of the ninth aspect includes but is not limited to the treatment of wounds and haemorrhage.

According to a tenth aspect of the present invention there is provided a method of making an in situ forming foam for use as a porous cavity filler and/or medicament in TNP therapy.

According to an eleventh aspect of the present invention there is provided a kit of parts, the kit comprising:
a container of a first constituent, Composition A, comprising an acidic solution of a polycationic polymer selected from the group comprising polymeric amines and poly saccharides;
a container of a second constituent, Composition B, comprising a component selected from the group comprising metal carbonates, metal bicarbonates, and mixtures of metal carbonates and bicarbonates;
means for mixing said Composition A and said Composition B together; and
means for applying the mixed Compositions to an intended site of application.

As discussed hereinabove, the means for storing Compositions A and B in the eleventh aspect of the present invention may be a dual barrelled syringe having appropriate volumes of each barrel according to the proportions of Compositions A and B required in the mixture.

The means of mixing the Compositions may be a static mixing head attached to or as an integral part of the syringe as may the means for applying the mixture to the intended site of application.

The Compositions in the kit according to the eleventh aspect of the present invention may be modified to include various additional therapeutic species as discussed hereinabove, for the treatment of a body or wound site. Alternatively, such additional therapeutic species may be provided in third or additional further containers in form of a multi-barrelled syringe wherein the contents of each barrel may be mixed as desired on expulsion from the containers.

In order that the present invention may be more fully understood, examples will now be described by way of illustration only.

### EXAMPLE 1

### Preparation of Acidic Chitosan Solution

Chitosan flakes (45 g) were added to a vigorously stirred volume of distilled water (1500 ml). To the vigorously stirred mixture was added glacial acetic acid (30 ml). The mixture rapidly became viscous and was left to stand unstirred for 48 hours. After this time, the viscous solution was homogeneous and transparent.

### EXAMPLE 2

### Preparation of Sodium Bicarbonate Suspension in Glycerol

Sodium bicarbonate (50 g) was stirred into glycerol (40 g), forming an homogenous suspension.

### EXAMPLE 3

### Loading of 1:10 Volume Ratio Double-Barrelled Syringe

Chitosan solution prepared in Example 1 (10 ml) and sodium bicarbonate suspension in glycerol prepared in Example 2 (1 ml) were loaded separately into the barrels of a 1:10 volume ratio double-barrelled syringe.

### EXAMPLE 4

### Preparation of Chitosan Foam

The loaded syringe prepared in Example 3 was discharged smoothly in a single ejection through a static mixing head onto siliconized release paper. The so-produced foam contained some expelled water and was homogeneous and mechanically robust. Mechanically robust in the context of this invention means able to withstand a surface compressive load exceeding 40 g/cm² without permanent structural disruption or permanent significant deformation.

### EXAMPLE 5

### Preparation of Sodium Bicarbonate Suspension in Glycerol

Finely milled (diameter <250 um) sodium carbonate powder (10 g) was stirred into glycerol (20 g), forming an homogeneous suspension.

### EXAMPLE 6

### Loading of 1:10 Volume Ratio Double-Barrelled Syringe

Chitosan solution prepared in Example 1 (10 ml) and sodium bicarbonate suspension in glycerol prepared in Example 5 (1 ml) were loaded separately into the barrels of a 1:10 volume ratio double-barrelled syringe.

### EXAMPLE 7

### Preparation of Chitosan Minimally Blown Foam

The loaded syringe prepared in Example 6 was discharged smoothly in a single ejection through a static mixing head onto siliconized release paper. The so-produced elastomer contained some expelled water and some trapped gas bubbles. The foam produced in this example was almost entirely closed cell and thus would not be suitable for a fluid-transmitting application such as TNP. This structure is useful however in void filling requiring greater mechanical rigidity than an open-celled foam—see Example 12. The foam was homogeneous and mechanically robust.

### EXAMPLE 8

### Demonstration of Wound Debris Clearing in the Absence of Blood

The loaded syringe prepared in Example 3 was discharged smoothly in a single ejection through a static mixing head onto a porcine wound cavity containing granular debris including gravel and soil particulates. After two minutes the foam, which filled the cavity, was removed by hand. The foam successfully recovered 80% of the debris from the wound cavity.

### EXAMPLE 9

### Demonstration of Wound Debris Clearing in the Presence of Blood

The loaded syringe prepared in Example 3 was discharged smoothly in a single ejection through a static mixing head onto a porcine wound cavity containing granular debris including gravel and soil particulates and excess blood. After two minutes the foam, which filled the cavity, was removed by hand. The foam successfully recovered over 80% of the debris from the wound cavity.

### EXAMPLE 10

### Demonstration of Blood Clotting Capability

The loaded syringe prepared in Example 3 was discharged smoothly in a single ejection through a static mixing head onto a polythene bag containing 10 ml fresh blood. After two minutes the foam was removed by hand. The foam successfully clotted and bound a layer of coagulum.

### EXAMPLE 11

### Use of Chitosan Foam in TNP Wound Therapy

The loaded syringe prepared in Example 3 was discharged smoothly in a single ejection through a static mixing head onto a porcine wound cavity. The cavity was overlayed with a sheet of CicaCare (Trade Mark of Smith and Nephew Medical Limited) silicone elastomeric dressing containing a central port. The dressing port was attached to a vacuum pump maintaining a pressure of 125 mmHg below ambient atmospheric pressure. Upon application of the vacuum, wound cavity contraction was observed and liquid was withdrawn from the wound cavity. At first, this liquid that was that expelled by the chitosan foam structure; this was followed by exudate from the porcine tissue (indicated by yellow colouration) demonstrating its fluid transmission capability. After one hour the vacuum was disconnected and the wound cavity returned to ambient pressure. The wound cavity was observed to relax. The CicaCare sheet was removed from the skin and the chitosan foam was removed, in a single piece and without difficulty, from the wound cavity. There was no significant tissue adherence. The foam was inspected and noted to be of open cell structure throughout and at the tissue-contacting margins. It was observed that the foam had moulded very well to the features of the wound cavity.

### EXAMPLE 12

### Demonstration of Ability to Fill Meniscal Defect

The loaded syringe prepared in Example 6 was discharged smoothly in a single ejection through a static mixing head into an 8 mm diameter meniscal defect created in a porcine cadaver hind leg knee joint. The elastomer was allowed to set for several minutes. The elastomer conformed well to the edges and surface of the defect.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method for the treatment of a wound site with a foam material, the method comprising:
mixing a first and a second component to form a mixed material, wherein the first component comprises an acidic solution of a polycationic polymer selected from the group consisting of a polymeric amines and polysaccharides, and wherein the second component is selected from the group consisting of a metal carbonate, a metal bicarbonate, and a mixture of a metal carbonate and a metal bicarbonate;
applying the mixed material to the wound site, wherein the mixed material forms a foamed composition;
sealing the wound site with a fluid tight sheet; and
applying topical negative pressure therapy to the wound site with a vacuum pump.

47. The method of claim 46, wherein the first and second components are stored in a dual-barreled syringe prior to mixing.

48. The method of claim 47, wherein mixing is effected through a static mixing head associated with the syringe.

49. The method of claim 46, wherein the first and second compositions are delivered and mixed at a rate that allows the mixed material to reach the wound site before significant foaming occurs.

50. The method of claim 46, wherein the wound site is sealed prior to applying the mixed material to the wound site.

51. The method of claim 46, wherein the wound site is connected to the vacuum pump via a port on the sheet.

52. The method of claim 46, wherein the first component includes a polysaccharide selected from the group consisting of chitin, a chitin derivative, chitosan, and a chitosan derivative.

53. The method of claim 46, further comprising including additional therapeutic species in the foamed composition, wherein the therapeutic species are selected from the group consisting of antimicrobial species including antibiotics and antibacterials, pain-killers, growth factors, protease inhibitors, biological products, and cells.

54. The method of claim 53, wherein the additional therapeutic species are incorporated into the foamed composition by mixing into the first component, mixing into the second component, or mixing into the first and second component.

55. The method of claim 46, wherein the negative pressure is applied through the foamed composition so that liquid is transmitted away from the wound site through the foamed composition.

56. The method of claim 55, wherein the foamed composition forms an open-celled foam.

57. A system for the treatment of a wound site, the system comprising:
a container of a first constituent, Composition A, comprising an acidic solution of a polycationic polymer selected from the group comprising polymeric amines and poly saccharides;
a container of a second constituent, Composition B, comprising a component selected from the group comprising metal carbonates, metal bicarbonates, and mixtures of metal carbonates and bicarbonates;
a sheet adapted to be placed over the wound site and capable of maintaining a fluid tight seal while under negative pressure; and
a vacuum pump configured to deliver negative pressure to the wound site;
wherein said Composition A and said Composition B are configured to be mixed together and applied to the wound site to form a foamed composition through which negative pressure is transmitted to the wound site.

58. The system of claim 57, wherein the sheet further comprises a port adapted to attach to the vacuum pump.

59. The system of claim 57, further comprising means for mixing Composition A and Composition B together.

60. The system of claim 57, further comprising means for applying the mixed Compositions A and B to the wound site.

61. The system of claim 57, further comprising a dual-barreled syringe provided with a static mixing head adapted for mixing the Compositions A and B.

62. The system of claim 57, further comprising a dual-barreled syringe adapted to apply the Compositions A and B to the wound site.
